(19) United States
(12) Patent Application Publication
(10) Pub. No.: US 2026/0232508 A1
(43) Pub. Date: Aug. 13, 2026

(54) Restraint System

(71) Applicant: The University of Toledo, Toledo, OH (US)

(72) Inventor: Shawn Monteiro, Toledo, OH (US)

(73) Assignee: The University of Toledo, Toledo, OH (US)

(21) Appl. No.: 19/530,619

(22) Filed: Feb. 5, 2026

Related U.S. Application Data
(60) Provisional application No. 63/755,514, filed on Feb. 7, 2025.

(51) Int. Cl. A61G 13/12; A61F 5/37

(52) U.S. Cl. CPC A61G 13/124; A61F 5/3776

(57) ABSTRACT

Restraint systems, and methods of using the same, are described. The restraint systems include a board and a restraint, such as a strap, where the board includes a plurality of cutouts. The plurality of cutouts includes a plurality of ovular cutouts and a plurality of X-shaped cutouts.

### RELATED APPLICATIONS

This application claims priority to U.S. Provisional Application No. 63/755,514, filed under 35 U.S.C. § 111(b) on Feb. 7, 2025, the disclosure of which is incorporated herein by reference in its entirety.

### STATEMENT REGARDING FEDERALLY SPONSORED RESEARCH

This invention was made with government support under Grant Number R25 EB029385 awarded by the National Institutes of Health. The government has certain rights in this invention.

### BACKGROUND

Hand restraints are commonly used in medical settings to limit the movement of a patient's hands or arms, particularly when there is a risk of self-harm, interference with medical procedures, or disruption of treatment. These devices are important in ensuring patient safety, preventing accidental removal of medical devices (such as intravenous lines or catheters), and maintaining control over patients who may be agitated or at risk of injuring themselves or others.

Conventional restraint devices, such as cloth or leather straps, are often used in medical environments; however, these restraints often have limitations related to patient comfort, east of application, and overall safety. Conventional restraints may lead to issues such as restricted circulation, bruising, skin irritation, or pressure ulcers if applied to tightly or left in place for extended periods. Additionally, many hand restraints in particular lack adjustable features to accommodate a wide range of hand sizes, which can be problematic in diverse patient populations including pediatric, elderly, or obese individuals. Furthermore, some other restraint devices currently on the market have issues with appendages slipping out of them and are expensive.

There is a need for new and improved restraint systems.

### SUMMARY

Provided herein is a restraint system comprising a board having a first surface and a second surface opposite the first surface, wherein the board includes a plurality of cutouts each extending through the board from the first surface to the second surface; and at least one restraint; wherein the plurality of cutouts includes a first cutout and a second cutout, wherein the first cutout is ovular and the second cutout is X-shaped.

In certain embodiments, the plurality of cutouts is arranged in a pattern of rows and columns, the board having a first row, a second row, a third row, and a fourth row, and a first column, a second column, a third column, and a fourth column. In particular embodiments, the plurality of cutouts includes a plurality of the first cutouts and a plurality of the second cutouts. In particular embodiments, the first row has a pattern of the plurality of cutouts comprising an alternating arrangement of one of the first cutouts and one of the second cutouts. In particular embodiments, successive ones of the first cutout are separated by a central support area. In particular embodiments, the central support area separates successive ones of the first cutout in adjacent columns.

In particular embodiments, the second cutouts are large enough to span multiple rows of the first cutouts.

In particular embodiments, at least two of the first row, the second row, the third row, and the fourth row includes an alternating arrangement of first cutouts and second cutouts. In particular embodiments, at least two of the first column, the second column, the third column, and the fourth column includes an alternating arrangement of first cutouts and second cutouts.

In particular embodiments, at least one of the first row, the second row, the third row, and the fourth row includes a third cutout and an alternating arrangement of first cutouts and second cutouts.

In certain embodiments, the board includes a plurality of protrusions extending from the second surface.

In certain embodiments, the restraint is a strap. In particular embodiments, the strap fits through each of the plurality of cutouts. In particular embodiments, the first cutout has a dimension which is larger than a width of the strap so as to allow the strap to fit through the first cutout. In particular embodiments, each of the first cutouts has a dimension which is larger than a width of the strap so as to allow the strap to fit through each of the first cutouts. In particular embodiments, the second cutout has a dimension which is larger than a width of the strap so as to allow the strap to fit through the second cutout. In particular embodiments, each of the second cutouts has a dimension which is larger than a width of the strap so as to allow the strap to fit through each of the second cutouts.

In certain embodiments, the restraint system further comprises a third cutout that is ovular, wherein the third cutout is longer than the first cutout. In particular embodiments, the restraint system comprises a plurality of the third cutouts. In particular embodiments, the plurality of the third cutouts is disposed along a perimeter of the board.

Further provided is a method of restraining a body part for surgery, the method comprising positioning a body part in a desired location and orientation on a board, wherein the board includes a plurality of cutouts extending through a thickness of the board, the plurality of cutouts including a plurality of ovular cutouts and a plurality of X-shaped cutouts; and securing the body part to the board in the desired location and orientation with a restraint, wherein the securing comprises inserting the restraint through at least one of the plurality of cutouts.

In certain embodiments, the securing comprises inserting the restraint through at least two of the plurality of cutouts. In certain embodiments, the restraint is a strap. In certain embodiments, the body part is a hand.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Photograph of a non-limiting example embodiment of a restraint system.

FIG. 2: Photograph of the non-limiting example embodiment of a restraint system being used to restrain a hand.

FIG. 3: Photograph of the non-limiting example embodiment of the restraint system shown in FIG. 1 with a restraint in the form of a strap extending through cutouts in the board of the restraint system.

FIG. 4: Illustration of a non-limiting example embodiment of a restraint system.

FIG. 5: Illustration of a non-limiting example embodiment of a restraint system.

FIG. 6: Illustration of a non-limiting example embodiment of a restraint system.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein is a reuseable restraint system that circumvents issues with conventional, one-time-use immobilizers currently used for various hand/carpal tunnel surgeries. The restraint system allows for a surgeon to focus less on appendages slipping out of a restraint than with devices currently on the market. Furthermore, the restraint system is cost efficient and allows for a wide variety of positioning of the anatomy being restrained. Although the following describes a reuseable hand restraint system, the restraint system is not limited to use with hands and can be used or modified to assist with other body parts such as, but not limited to, knees, feet, torsos, and heads.

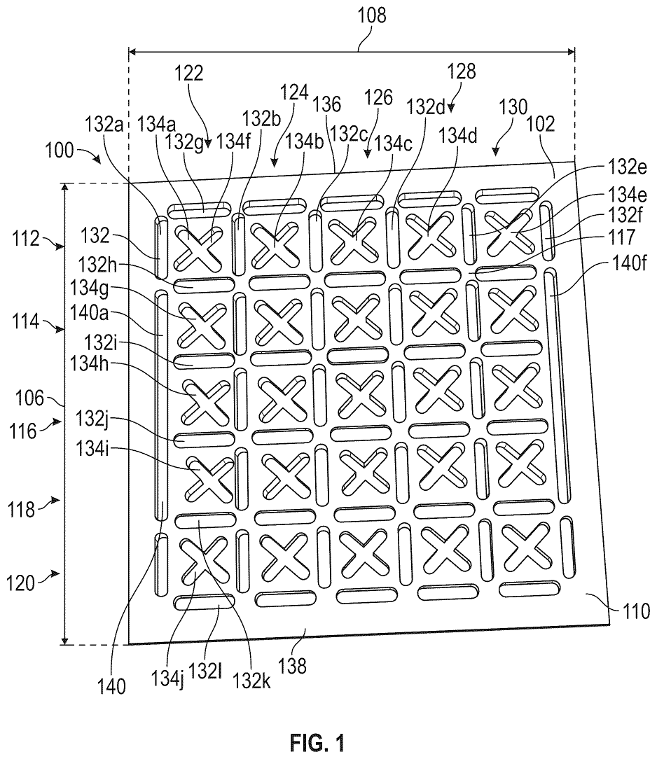

Referring now to FIGS. 1-4, embodiments of a restraint system 100 are depicted. The restraint system 100 includes a board 102 and at least one restraint 104. The board 102 is a cuboid that has a length 106, a width 108, and a thickness 111; however, the board 102 can be any desired shape such as, but not limited to, a cylinder, a cube, a hexagonal prism, or the like. The length 106 and the width 108 can be approximately the same; however, in alternative embodiments, the length 106 and the width 108 are different. The thickness 111 is less than both the length 106 and the width 108.

The board 102 can be made from any suitable material and by any suitable process. For example, the board 102 can be made from polyetheretherketone (PEEK). It is preferred to use a material that is lighter than steel, but steel can nonetheless be used to make the board 102. Further, the board 102 can be made by using injection molding. The board 102 can also be 3D printed. The board 102 can also have a thickness 111 that is approximately 5-10 mm thick; however, the board 102 may be any suitable thickness. However, other materials and processes are possible and encompassed within the scope of the present disclosure.

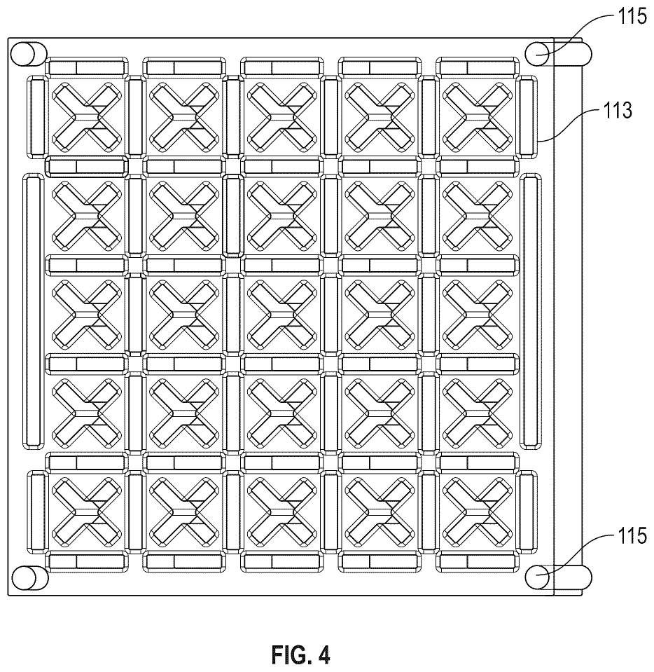

Referring to FIG. 1 and FIG. 4, the board 102 has a first surface 110 and a second surface 113 opposite of the first surface 110, extending for the thickness 111 between the first surface 110 and the second surface 113. In one non-limiting example, the board 102 is about 9 inches long and about 9 inches wide. However, any dimensions of the board 102 suitable for the intended use of the board 102 can be used. In the illustrated embodiment, the dimension between the first surface 110 and the second surface 113 (i.e., the thickness 111 of the board 102) is approximately 0.5 inches. However, the board 102 can have many other thicknesses, and such other thicknesses are encompassed within the scope of the present disclosure.

Referring to FIG. 1, the board 102 has a plurality of cutouts arranged in a plurality of rows and a plurality of columns. In the embodiment shown in FIG. 1, there are five rows including a first row 112, a second row 114, a third row 116, a fourth row 118, and a fifth row 120, and five columns including a first column 122, a second column 124, a third column 126, a fourth column 128, and a fifth column 130. Each column of the plurality of columns and each row of the plurality of rows has a plurality of cutouts extending from the first surface 110 to the second surface 113 (i.e., extending through the entire thickness 111 of the board 102).

Referring still to FIG. 1, each row and each column includes at least two types of cutouts: a first cutout 132 and a second cutout 134. The first cutout 132 and the second cutout 134 may alternate in the pattern of each row and in the pattern of each column. The space between successive second cutouts 134 in each row includes a first cutout 132. The space between successive first cutouts 132 in each row includes a second cutout 134. Each first cutout 132 and each second cutout 134 is configured to accept a restraint, such as a strap, for restraining anatomy by securing the anatomy to the board 102 in a desired position and orientation.

Referring still to FIG. 1, the first cutout 132 is an ovular cutout. Each first cutout 132 may extend either a direction of from a first side 136 to a second side 138 or perpendicular to such direction. The first cutout 132 may extend, as a non-limiting example, approximately 1.25 inches and have a width of, as a non-limiting example, approximately 0.25 inches. However, other dimensions are possible and encompassed within the scope of the present disclosure.

The second cutout 134 is an X-shaped cutout. The X-shaped cutout can be formed by two ovular segments intersecting at a center point to form an X-shape. In the embodiment depicted in FIG. 1, each of the ovular segments of the second cutout 134 is shorter in length than the ovular first cutout 132. In this manner, the X-shaped second cutout 134 can fit within an area bounded by adjacent ovular first cutouts 132, as seen in FIG. 1. More specifically, in the embodiment depicted in FIG. 1, each second cutout 134 of the first row 112 fits within an area bounded by successive first cutouts 132 of the first row 112 and successive first cutouts 132 in one of the columns 122, 124, 126, 128, 130. Repeating this pattern creates central support areas 117 between diagonally adjacent second cutouts 134, as seen in FIG. 1. The central support areas 117 are bounded by two adjacent first cutouts 132 in successive columns and two adjacent first cutouts 132 in successive rows. The central support areas 117 provide structural integrity to the board 102.

The X-shaped second cutouts 134 provide versatility, giving surgeons the most options when securing a hand in place on the board 102 for surgery. The X-shaped second cutouts 134 also allow for diagonal and straight routed straps to be used in any combination or configuration desired by the surgeon. The X-shaped second cutouts 134 also prevent such straps from becoming twisted when securing anatomy (such as a hand) to the board 102. The X-shaped second cutouts 134 also make the system 100 cheaper to manufacture because less material is needed to produce the board 102 without sacrificing structural integrity. The combination of ovular first cutouts 132, X-shaped second cutouts 134, and central support areas 117 allows for minimizing the amount of material used to manufacture the board 102 while not sacrificing the structural integrity of the board 102.

In the embodiment shown in FIG. 1, the first row 112 has a pattern of cutouts that includes, in order: a first cutout 132a extending a height of the first row 112, a second cutout 134a spanning a distance along the length of the first row 112, a first cutout 132b extending the height of the first row 112, a second cutout 134b spanning a distance along the length of the first row 112, a first cutout 132c extending a height of the first row 112, a second cutout 134c spanning a distance along the length of the first row 112, a first cutout 132d extending the height of the first row 112, a second cutout 134d spanning a distance along the length of the first row 112, a first cutout 132e extending the height of the first row 112, a second cutout 134e spanning a distance along the length of the first row 112, and a first cutout 132f extending the height of the first row 112.

In the embodiment shown in FIG. 1, the second row 114 includes not only first cutouts 132 and second cutouts 134, but also includes a third cutout 140. The third cutout 140 is similar to the first cutout 132 in that it is an ovular cutout, but the third cutout 140 is longer (i.e., spans more of the length 106) than the first cutout 132. In some non-limiting examples, the third cutout 140 has a length of approximately 4.45 inches and a width of approximately 0.25 inches. The third cutout 140 extends over the height of multiple rows, allowing the use of bigger straps. The bigger straps can be beneficial for strapping the board 102 down to a table or other surface.

In the embodiment shown in FIG. 1, the second row 114 has a pattern of cutouts which includes: a third cutout 140a extending over a height of the second row 114, the third row 116, and the fourth row 118; a second cutout 134 spanning a distance along the length of the second row 114; a first cutout 132 extending the height of the second row 114; a second cutout 134 spanning a distance along the length of the second row 114; a first cutout 132 extending the height of the second row 114; a second cutout 134c spanning a distance along the length of the second row 114; a first cutout 132 extending the height of second row 114; a second cutout 134 spanning a distance along the length of the second row 114; a first cutout 132 extending the height the second row 114; a second cutout 134e spanning a distance along the length of the second row 114; and a third cutout 140f extending the height of the second row 114, the third row 116, and the fourth row 118.

In the embodiment depicted in FIG. 1, the third row 116 includes the same pattern of cutouts as the second row 112, beginning and ending with a third cutout 140 and having alternating second and first cutouts 132, 134 therebetween. The fourth row 118 also includes the same pattern of cutouts as the second row 112, beginning and ending with a third cutout 140 and having alternating second and first cutouts 132, 134 therebetween. The fifth row 120, however, includes the same pattern of cutouts as the first row 112, beginning and ending with a first cutout 132 and having alternating second and first cutouts 132, 134 therebetween.

In the embodiment shown in FIG. 1, the first column 122 includes an alternating pattern of first and second cutouts 132, 134. The alternating pattern of first and second cutouts 132, 134 includes a first cutout 132g, a second cutout 134f, a first cutout 132h, a second cutout 134g, a first cutout 132i, a second cutout 134h, a first cutout 132j, a second cutout 134i, a first cutout 132k, a second cutout 134j, and a first cutout 132l. In fact, each of the first column 122, the second column 124, the third column 126, the fourth column 128, and the fifth column 130 includes the same alternating pattern of first and second cutouts 132, 134, beginning and ending with a first cutout 132. For clarity, each of the first column 122, the second column 124, the third column 126, the fourth column 128, and the fifth column 130 includes second cutouts 134 from each of the first row 112, the second row 114, the third row 116, the fourth 118, and the fourth row 120, with first cutouts 132 therebetween. In the embodiment shown in FIG. 1, each of the first column 122, the second column 124, the third column 126, the fourth column 128, and the fifth column 130 has the same pattern of cutouts. However, this is not strictly necessary and other arrangements or patterns are possible and encompassed within the scope of the present disclosure.

Referring now to FIG. 4, the second surface 113 of the board 102 may include four protrusions 115 extending therefrom. The protrusions 115 may be cylindrical in shape and may, for example, each have a radius that is approximately 0.2 inches. However, other radii are possible and encompassed within the scope of the present disclosure. Each protrusion 115 is configured to provide space between the board 102 and a surface on which the board 102 rests. In other words, the protrusions 115 act as feet for the board 102 to facilitate resting the board 102 on a surface. The protrusions 115 also allow clearance for the strap on the underside of the board 102. Although the protrusions 115 are described as being cylindrical, any suitable shaped protrusions 115 can be used. Furthermore, although FIG. 4 depicts the second surface 113 including four protrusions 115, alternative embodiments can include any number of protrusions 115 including, but not limited to, two, three, five, six, or seven. In some non-limiting examples, each of the protrusions 115 extends approximately 0.2 inches from the second surface 113. However, the protrusions 115 can extend any suitable distance from the second surface 113.

Optionally, a sterile cloth may be draped underneath the board 102. Furthermore, a padding may be disposed on the first surface 110 of the board 102 to prevent nerve damage caused from a patient's nerves being pressured against the surface of the board 102. However, these features are not strictly necessary.

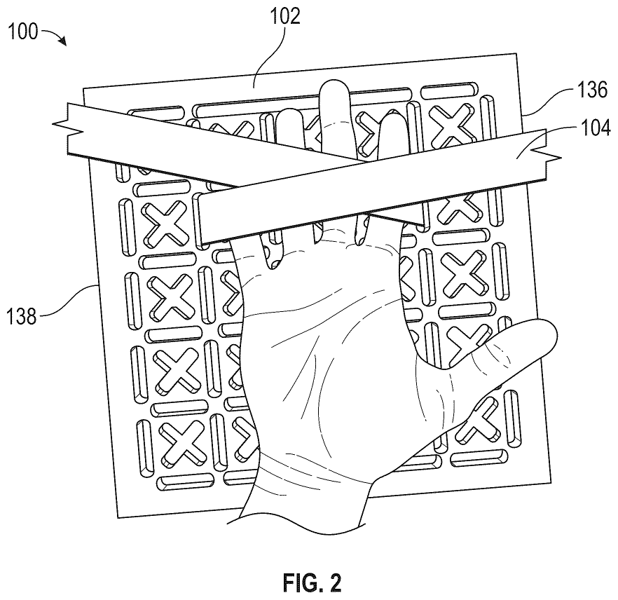

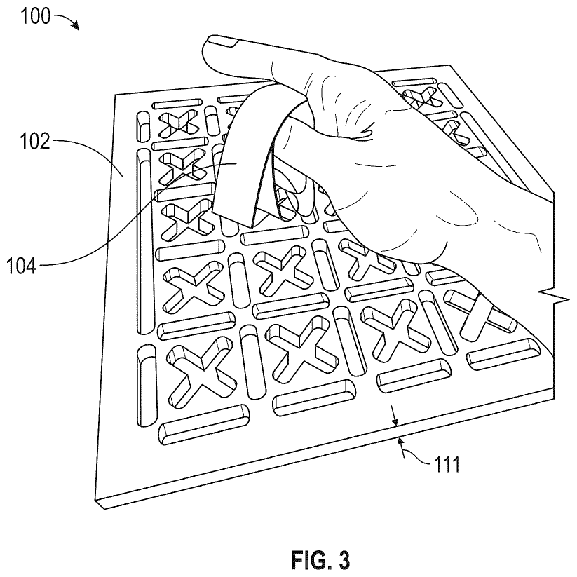

Referring now to FIGS. 2-3, the restraint system 100 includes a restraint 104. Although the restraint 104 is illustrated as a strap having a hook and loop fastening system, any means of securing a patient's hand or other anatomy to the board 102 can be used, provided that the restraint 104 fits through the cutouts 132, 134, 140, 142. The restraint 104 is configured to extend through the first cutout 132, the second cutout 134, and the third cutout 140. In the case of a strap, the strap may have a width which is smaller than the length of the ovular first cutouts 132 and smaller than the width of each ovular segment of the X-shaped second cutouts 134, or otherwise be foldable so as to be able to fit through the cutouts 132, 134. The restraint 104 can be, for example, an autoclavable and reusable strap or, as another example, a disposable hook and latch strap. Straps can be beneficial as the restraint 104 because in some procedures the anatomy being restrained needs to be quickly released, and straps provide a means for quickly removing the anatomy from the board 102.

In one non-limiting example use of the restraint system 100, a hand of a patient is placed on the first surface 110 of the board 102 and then positioned into a desired position and orientation on the board 102. One or more restraints 104 are used to secure the hand in place on the board 102 and in the desired position and orientation, using one or more of the cutouts 132, 134, 140 to secure the restraint 104 and hand on the board 102. The restraint 104 can be fastened to the board 102 by any suitable means, but it is advantageous to run the restraint 104 through multiple of the cutouts 132, 134, 140, such as one on each side of the patient's hand, in order to securely stabilize the patient's hand on the board 102. The restraint 104 can be adjusted during the procedure, so as to change the position of the patient's hand. The arrangement of cutouts 132, 134, 140 in the board 102 is beneficial in this regard because the surgeon has substantially no restrictions on how to secure the patient's hand to the board 102. The patient's hand can be secured in any desired position and orientation during the procedure.

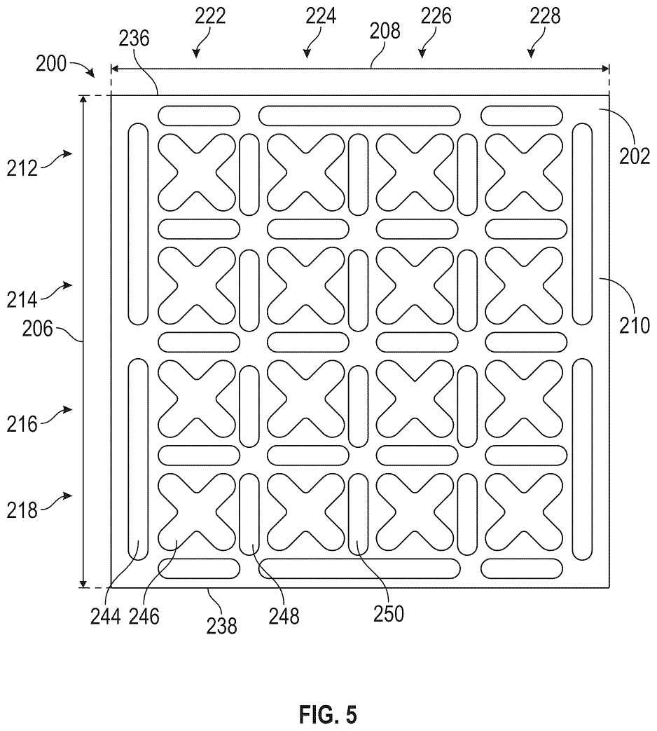

Referring now to FIG. 5, another embodiment of a restraint system 200 is depicted. The restraint system 200 includes a board 202 and at least one restraint. The board 202 is a cuboid that has a length 206, a width 208, and a height. However, the board 202 can be any desired shape such as, but not limited to, a cylinder, a cube, a hexagonal prism, or the like. The length 206 and the width 208 can be approximately the same; however, in alternative embodiments, the length 206 and the width 208 are different. The thickness is less than both the length 206 and the width 208.

Referring still to FIG. 5, the board 202 has a first surface 210 and a second surface opposite of the first surface 210. The board 202 has a plurality of cutouts positioned by a plurality of rows and a plurality of columns. The plurality of cutouts includes four rows including a first row 212, a second row 214, a third row 216, and a fourth row 218, and four columns including a first column 222, a second column 224, a third column 226, and a fourth column 228. Each cutout in the plurality of cutouts extends from the first surface 210 to the second surface (i.e., through the entire thickness of the board 202). As with the previous embodiments, the plurality of cutouts includes a plurality of first cutouts 248, which are ovular, and a plurality of second cutouts 246, which are X-shaped. The plurality of cutouts also includes a plurality of third cutouts 244, which are ovular but longer (i.e., span more of the length 206) than the first cutouts 248. The plurality of cutouts also includes a plurality of fourth cutouts 250, which are also ovular but wider (i.e., span more of the width 208) than the first cutouts 248. The longer third cutouts 244 are positioned around the perimeter, at the start and end of the rows 212, 214, 216, 218, and also at the start and end of some of the columns 224, 226, although not the columns 222, 228.

As noted, the restraint system 200 includes a restraint. The restraint can be the same as in the previous embodiments. For example, the restraint can be a strap having a hook and loop fastening system; however, any means of securing anatomy, such as a patient's hand, to the board 202 can be used. Advantageously, the restraint should have a width which is smaller than at least one dimension of the cutouts, to ensure that the restraint can fit through the cutouts. Once the anatomy (e.g., a hand) is in a desired position and orientation, the one or more restraints can be disposed through at least one of the plurality of cutouts in order to secure the anatomy to the board 202. The one or more restraints can then be fastened by any desired means to hold the anatomy in place while a surgical procedure is performed. The shape and arrangement of cutouts described herein is beneficial for securing anatomy, such as a patient's hand, in any desired position.

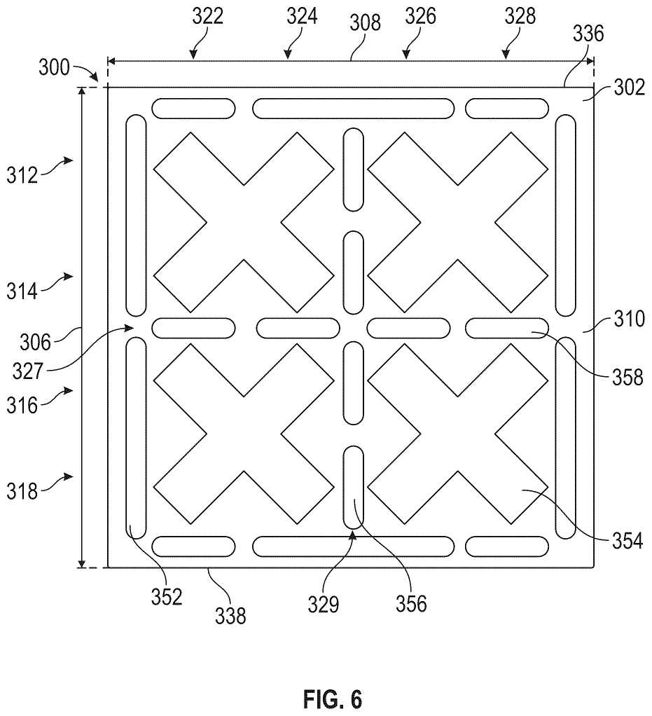

Referring now to FIG. 6, a further embodiment of a restraint system 300 is depicted. The restraint system 300 includes a board 302 and at least one restraint. The board 302 is a cuboid that has a length 306, a width 308, and a height. However, the board 302 can be any desired shape such as, but not limited to, a cylinder, a cube, a hexagonal prism, or the like. In the illustrated embodiment, the length 306 and the height are approximately the same; however, in alternative embodiments, the length 306 and the height are different. The width 308 is less than both the length 306 and the height.

Referring still to FIG. 6, the board 302 has a first surface 310 and a second surface opposite of the first surface 310. The board 302 has a plurality of cutouts positioned in a plurality of rows and a plurality of columns. The plurality of cutouts includes first cutouts 358, which are ovular, second cutouts 354, which are X-shaped, third cutouts 352, which are ovular but longer (i.e., span more of the length 306) than the first cutouts 358, and fourth cutouts 356 which are ovular but wider (i.e., span more of the width 308) than the first cutouts 358. However, in this embodiment, the second cutouts 354 are each large enough to span two rows 312, 314 or 316, 318 of the plurality of cutouts. Each of the second cutouts 354 is also large enough to span two columns 322, 324 or 326, 328 of the plurality of cutouts. As with previous embodiments, the perimeter of the board 302 includes an arrangement of first cutouts 356 and third cutouts 352. The plurality of cutouts includes a center row 327 consisting of only first cutouts 358 and a center column 329 consisting of only fourth cutouts 356.

It should be appreciated that many different arrangements of ovular and X-shaped cutouts can be utilized in the board 102, 202, 302 of the restraint system 100, 200, 300 described herein. As long as the restraint 104, such as a strap, has a width which is smaller than at least one dimension of the cutouts in the plurality of the cutouts, or is foldable so as to fit through the cutouts, then the arrangement of ovular and X-shaped cutouts provides for a highly useful and versatile restraint system.

Certain embodiments of the devices, systems, and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices, systems, and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A restraint system comprising:
a board having a first surface and a second surface opposite the first surface, wherein the board includes a plurality of cutouts each extending through the board from the first surface to the second surface; and
at least one restraint;
wherein the plurality of cutouts includes a first cutout and a second cutout, wherein the first cutout is ovular and the second cutout is X-shaped.

2. The restraint system of claim 1, wherein the plurality of cutouts is arranged in a pattern of rows and columns, the board having a first row, a second row, a third row, and a fourth row, and a first column, a second column, a third column, and a fourth column.

3. The restraint system of claim 2, wherein the plurality of cutouts includes a plurality of the first cutouts and a plurality of the second cutouts.

4. The restraint system of claim 3, wherein the first row has a pattern of the plurality of cutouts comprising an alternating arrangement of one of the first cutouts and one of the second cutouts.

5. The restraint system of claim 3, wherein successive ones of the first cutout in adjacent columns are separated by a central support area.

6. The restraint system of claim 1, wherein the board includes a plurality of protrusions extending from the second surface.

7. The restraint system of claim 1, wherein the restraint is a strap.

8. The restraint system of claim 7, wherein the strap fits through each of the plurality of cutouts.

9. The restraint system of claim 7, wherein the first cutout has a dimension which is larger than a width of the strap so as to allow the strap to fit through the first cutout.

10. The restraint system of claim 7, wherein each of the first cutouts has a dimension which is larger than a width of the strap so as to allow the strap to fit through each of the first cutouts.

11. The restraint system of claim 7, wherein the second cutout has a dimension which is larger than a width of the strap so as to allow the strap to fit through the second cutout.

12. The restraint system of claim 7, wherein each of the second cutouts has a dimension which is larger than a width of the strap so as to allow the strap to fit through each of the second cutouts.

13. The restraint system of claim 3, wherein the second cutouts are large enough to span multiple rows of the first cutouts.

14. The restraint system of claim 1, further comprising a third cutout that is ovular, wherein the third cutout is longer than the first cutout.

15. The restraint system of claim 14, comprising a plurality of the third cutouts disposed along a perimeter of the board.

16. The restraint system of claim 3, wherein at least two of the first row, the second row, the third row, and the fourth row includes an alternating arrangement of first cutouts and second cutouts.

17. The restraint system of claim 16, wherein at least two of the first column, the second column, the third column, and the fourth column includes an alternating arrangement of first cutouts and second cutouts.

18. The restraint system of claim 3, wherein at least one of the first row, the second row, the third row, and the fourth row includes a third cutout and an alternating arrangement of first cutouts and second cutouts.

19. A method of restraining a body part for surgery, the method comprising:
positioning a body part in a desired location and orientation on a board, wherein the board includes a plurality of cutouts extending through a thickness of the board, the plurality of cutouts including a plurality of ovular cutouts and a plurality of X-shaped cutouts; and
securing the body part to the board in the desired location and orientation with a restraint, wherein the securing comprises inserting the restraint through at least one of the plurality of cutouts.

20. The method of claim 19, wherein the body part is a hand.
